(19) United States
(12) Patent Application Publication
(10) Pub. No.: US 2011/0229444 A1
(43) Pub. Date: Sep. 22, 2011

(54) Neuregulin And Cardiac Stem Cells

(75) Inventor: Mingdong Zhou, Shanghai (CN)

(73) Assignee: Zensun (Shanghai) Science & Technology Limited, Shanghai (CN)

(21) Appl. No.: 13/131,668

(22) PCT Filed: Nov. 9, 2009
(86) PCT No.: PCT/CN2009/001237

§ 371 Date: May 27, 2011

Related U.S. Application Data
(60) Provisional application No. 61/118,563, filed on Nov. 28, 2008.

(51) Int. Cl. A61K 35/34; C12N 5/00; A61K 38/18; A61P 9/00

(52) U.S. Cl. 424/93.7; 435/377; 514/9.6

(57) ABSTRACT

The present invention relates to compositions and methods for inducing cardiomyogenesis in mammalian cells, particularly embryonic stem cells in vitro and in vivo.

This application is the national stage application of International Application No. PCT/CN2009/001237, filed Nov. 9, 2009, which claims the priority from U.S. Provisional Application No. 61/118,563, filed Nov. 28, 2008, the contents of which are hereby incorporated by reference in their entirety.

### 1. FIELD OF THE INVENTION

The present invention relates to use of neuregulin for inducing cardiomyogenesis in mammalian cells, particularly embryonic stem cells in vitro and in vivo.

### 2. BACKGROUND OF THE INVENTION

Cardiac (ventricular) hypertrophy is an important adaptive physiological response to increased stress or demands for cardiac work. One of the early cellular changes that occurs after a stimulus for hypertrophy is the synthesis of mitochondria and expansion of myofibrillar mass (wall thickening) with a proportional increase in the size of individual cells, but no (or minimal) increase in the number of cells.

When the ventricle is stressed, the initial response is an increase in sarcomere length. This is followed by an increase in the total muscle mass. When the overload is severe; myocardial contractility becomes depressed. In its mildest form, this depression is manifested by a reduction in the velocity of shortening of unloaded myocardium or by a reduction in the rate of force development during isometric contraction. As myocardial contractility becomes further depressed, a more extensive reduction in the velocity of shortening of unloaded myocardium occurs, now accompanied by a decline in isometric force development and shortening. At this point, circulatory compensation may still be provided by cardiac dilation and an increase in muscle mass, which tend to maintain wall stress at normal levels. As contractility falls further, overt congestive heart failure, reflected in a depression of cardiac output and work and/or an elevation of ventricular end-diastolic volume and pressure at rest, supervenes.

The transition from hypertrophy to heart failure is characterized by several alterations in cellular organization. For example, normal hypertrophic cells have a large size with increased and well organized contractile units, as well as strong cell-cell adhesions. In contrast, pathologically hypertrophic cells, which also have large size and accumulation of proteins, display disorganization of contractile proteins (disarray of sarcomeric structures) and poor cell-cell adhesions (disarray of myofibers). Thus, in pathological hypertrophy, the increased size and accumulation of contractile proteins are associated with disorganized assembly of sarcomeric structures and a lack of robust cell-cell interactions.

Heart failure affects approximately five million Americans, and more than 550,000 new patients are diagnosed with the condition each year. Current drug therapy for heart failure is primarily directed to angiotensin-converting enzyme (ACE) inhibitors, which are vasodilators that cause blood vessels to expand, lowering blood pressure and reducing the heart's workload. While the percent reduction in mortality has been significant, the actual reduction in mortality with ACE inhibitors has averaged only 3%-4%, and there are several potential side effects.

ACE inhibitors have also been administered in combination with other drugs such as digitalis, which increases the force of the heart's contractions, and/or a diuretic, which helps relieve the heart's workload by causing the kidneys to remove more sodium and water from the bloodstream. However, at least one study demonstrated no difference in survival associated with the use of digitalis compared with placebo in patients with Class II-III heart failure. Additionally, diuretics can improve some symptoms of heart failure but it is not suitable as a sole treatment.

Additional limitations are associated with other options for preventing or treating heart failure. For example, heart transplantation is clearly more expensive and invasive than drug treatment, and it is further limited by the availability of donor hearts. Use of mechanical devices, such as biventricular pacemakers, are similarly invasive and expensive. Thus, there has been a need for new therapies given the deficiencies in current therapies.

One promising new therapy involves administration of neuregulin (hereinafter referred to as “NRG”) to a patient suffering from or at risk of developing heart failure. NRGs comprise a family of structurally related growth and differentiation factors that include NRG1, NRG2, NRG3 and NRG4 and isoforms thereof. For example, over 15 distinct isoforms of NRG1 have been identified and divided into two large groups, known as α- and β-types, on the basis of differences in the sequence of their essential epidermal growth factor (EGF)-like domains.

NRGs bind to the EGF receptor family, which comprises EGFR, ErbB2, ErbB3 and ErbB4, each of which plays an important role in multiple cellular functions, including cell growth, differentiation and survival. They are protein tyrosine kinase receptors, consisting of an extracellular ligand-binding domain, transmembrane domain and cytoplasmic tyrosine kinase domain. After NRG binds to the extracellular domain of ErbB3 or ErbB4, it induces a conformational change that leads to heterodimer formation between ErbB3, ErbB4 and ErbB2 or homodimer formation between ErbB4 itself, which results in phosphorylation of the receptors' C-terminal domain inside the cell membrane. The phosphorylated intracellular domain then binds additional signal proteins inside the cell, activating the corresponding downstream AKT or ERK signaling pathway, and inducing a series of cell reactions, such as stimulation or depression of cell proliferation, cell differentiation, cell apoptosis, cell migration or cell adhesion. Among these receptors, mainly ErbB2 and ErbB4 are expressed in the heart.

It has been shown that the EGF-like domains of NRG1, ranging in size from 50 to 64-amino acids, are sufficient to bind to and activate these receptors. Previous studies have shown that neuregulin-1β (NRG-1β) can bind directly to ErbB3 and ErbB4 with high affinity. The orphan receptor, ErbB2, can form a heterodimer with ErbB3 or ErbB4 with higher affinity than ErbB3 or ErbB4 homodimers. Research in neural development has indicated that the formation of the sympathetic nervous system requires an intact NRG-1β, ErbB2 and ErbB3 signaling system. Targeted disruption of the NRG-1β or ErbB2 or ErbB4 led to embryonic lethality due to cardiac development defects. Recent studies also highlighted the roles of NRG-1β, ErbB2 and ErbB4 in the cardiovascular development as well as in the maintenance of adult normal heart function. NRG-1β has been shown to enhance sarcomere organization in adult cardiomyocytes. The short-term administration of a recombinant NRG-1β EGF-like domain significantly improves or protects against deterioration in myocardial performance in three distinct animal models of heart failure. More importantly, NRG-1β significantly prolongs survival of animals suffering from heart failure. These effects make NRG-1β promising as a broad spectrum therapeutic or lead compound for heart failure due to a variety of common diseases.

Pluripotent embryonic stem (ES) cells represent a possible unlimited source of functional cardiomyocytes. Such cardiomyocytes would likely facilitate the therapeutic application of ES cells in heart disease, as well as provide important tools for probing the molecular mechanism of cardiomyocyte differentiation and heart development. Several recent studies have provided evidence for cardiac progenitor cells that have the potential to differentiate into all three of the major cell types of the heart: cardiac myocytes, smooth muscle cells and endothelia cells. See, e.g., Boheler et al., Circ. Res., 91:189-201 (2002). Kattman et al., 2006, Dev Cell, 11:723-732, 2006; Moretti et al., 2006, Cell, 127:1151-1165; Wu et al., Cell, 127:1137-1150; Garry et al., 2006, Cell, 127:1101-1104, 2006. To date, however, the in vitro differentiation of ES cells into cardiomyocytes involves a poorly defined, inefficient and relatively non-selective process.

Thus, the art recognizes a need for compositions and methods for inducing and directing the differentiation of ES cells into cardiomyocytes. There is a particular need for small molecules that can induce in vivo and in vitro differentiation of ES cells into cells of a myocardial lineage. This invention satisfies these and other needs.

### 3. BRIEF DESCRIPTION OF THE FIGURES

FIG. 1 compares the sections of injured heart treated with vehicle or neuregulin. FIG. 1a and FIG. 1b shows sections of infracted heart treated with vehicle, while FIG. 1c and FIG. 1d shows sections of infracted heart treated with neuregulin.

### 4. SUMMARY OF THE INVENTION

The present invention provides use of neuregulin for inducing and directing the differentiation of ES cells into cells of a myocardial lineage.

Any neuregulin (e.g., NRG-1, NRG-2, NRG-3 and NRG-4 and isoforms thereof) protein, peptide or fragment can be used in the present invention. In certain embodiments, neuregulin comprises the EGF-like domain encoded by NRG-1. In some embodiments, neuregulin comprises the amino acid sequence of SEQ ID NO:1.

In another embodiment, the present invention provides methods of inducing cardiomyogenesis. In certain embodiments, mammalian cells are contacted with neuregulin, whereupon the mammalian cell differentiates into a cell of a myocardiac lineage. The step of contacting can be in vivo or in vitro. In view of its ability to induce cardiomyogenesis, neuregulin, in certain embodiments, is useful for treating cardiac muscle disorders, such as heart failure, cardiomyopathy and arrhythmia, and for repairing heart muscle tissue damage resulting from a heart attack, for example.

The present invention provides methods of treating cardiac muscle disorders by contacting a mammalian cell with neuregulin, whereupon the mammalian cell differentiates into a cell of a myocardiac lineage. The mammalian cell may be further contacted with other compounds or proteins favorable to cardiomyogenesis. If the mammalian cell is contacted with neuregulin in vitro, the differentiated cells can be administered to a subject with a treatable disorder, thereby treating the disorder. In some embodiments, the mammalian cell is attached to a solid support (e.g., a three-dimensional matrix or a planar surface) or injected to the damaged sites of myocardium.

In some embodiments, the mammalian cell is contacted with neuregulin in vivo. If the mammalian cell is contacted with neuregulin in vivo, the step of contacting may be via any route known to one skilled in the art without limitation, including oral, intravenous, subcutaneous, or intraperitoneal administration of neuregulin to the mammal.

The differentiation of the mammalian cell into a cell of a myocardiac lineage can be detected by any technique known in the art. In some embodiments, the differentiation of the mammalian cell into a cell of a myocardioblast is detected by detecting expression of a cardiomyogenesis marker gene, e.g., atrial natriuretic factor (“ANF”). In other embodiments, the differentiation of the mammalian cell into a cell of a myocardiac lineage is detected by detecting expression of a cardiac muscle cell-specific transcription factor (e.g., MEF2 or Nkx2.5 or the homeodomain transcription factor HOP). In other embodiments, the differentiation of the mammalian cell into a cell of a myocardiac lineage is detected by detecting expression of a cardiac muscle specific gene (e.g., myosin light chain 2V or eHAND). In still other embodiments, the differentiation of the mammalian cell into a cell of a myocardiac lineage is detected by detecting expression of a cardiac specific gene, such as GATA-4, or by the expression of a gene involved in cardiac muscle contractibility, such as the sarcomeric myosin heavy chain (MHC). In further embodiments, the differentiation may be detected by observing the beating of cardiac muscle using standard techniques well-known to those in the art.

In some embodiments, the mammalian cell is a stem cell (e.g., an embryonic stem cell or an embryonic carcinoma cell). In some embodiments, the stem cell is isolated from a mouse (e.g., a murine undifferentiated R1 embryonic stem cell or a murine carcinoma P19 cell) or from a primate (e.g., a human).

### 5. DETAILED DESCRIPTION OF THE INVENTION

### 5.1 Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” mean “at least one” or “one or more” unless the context clearly dictates otherwise.

The term “about” or “approximately” means within 20%, preferably within 10%, and more preferably within 5% (or 1% or less) of a given value or range.

As used herein, “administer” or “administration” refers to the act of injecting or otherwise physically delivering a substance as it exists outside the body (e.g., neuregulin) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

As used herein, “an effective amount” means an amount of neuregulin or compositions comprising neuregulin that when, administered to a subject for treating a disease is sufficient to effect such treatment for the disease. An effective amount can vary depending on, inter alia, the neuregulin used, the disease and its severity, and the age, weight, etc. of the subject to be treated. In some embodiments, an effective amount means an amount of neuregulin sufficient to reduce pulmonary capillary wedge pressure.

As used herein, “neuregulin” or “NRG” used in the present invention refers to proteins or peptides that can bind and activate ErbB2, ErbB3, ErbB4 or combinations thereof, including but not limited to all neuregulin isoforms, neuregulin EGF domain alone, polypeptides comprising neuregulin EGF-like domain, neuregulin mutants or derivatives, and any kind of neuregulin-like gene products that also activate the above receptors as described in detail below. In preferred embodiments, neuregulin used in the present invention binds to and activates ErbB2/ErbB4 or ErbB2/ErbB3 heterodimers. Neuregulin also includes NRG-1, NRG-2, NRG-3, and NRG-4 proteins, peptides, fragments and compounds that mimic the activities of neuregulin. Neuregulin used in the present invention can activate the above ErbB receptors and modulate their biological reactions, e.g., stimulate breast cancer cell differentiation and milk protein secretion; induce the differentiation of neural crest cell into Schwann cell; stimulate acetylcholine receptor synthesis in skeletal muscle cell; and/or improve cardiocyte differentiation, survival and DNA synthesis. Neuregulin also includes those variants with conservative amino acid substitutions that do not substantially alter their biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Neuregulin protein encompasses neuregulin protein and peptide. Neuregulin nucleic acid encompasses neuregulin nucleic acid and neuregulin oligonucleotide.

As used herein, “epidermal growth factor-like domain” or “EGF-like domain” refers to a polypeptide motif encoded by the neuregulin gene that binds to and activates ErbB2, ErbB3, ErbB4, or combinations thereof, and bears a structural similarity to the EGF receptor-binding domain as disclosed in WO 00/64400, Holmes et al., Science, 256:1205-1210 (1992); U.S. Pat. Nos. 5,530,109 and 5,716,930; Hijazi et al., Int. J. Oncol., 13:1061-1067 (1998); Chang et al., Nature, 387:509-512 (1997); Carraway et al., Nature, 387:512-516 (1997); Higashiyama et al., J. Biochem., 122:675-680 (1997); and WO 97/09425, the contents of which are all incorporated herein by reference. In certain embodiments, EGF-like domain binds to and activates ErbB2/ErbB4 or ErbB2/ErbB3 heterodimers. In certain embodiments, EGF-like domain comprises the amino acid sequence of the receptor binding domain of NRG-1. In some embodiments, EGF-like domain comprises the amino acid sequence corresponding to amino acid residues 177-226, 177-237, or 177-240 of NRG-1. In certain embodiments, EGF-like domain comprises the amino acid sequence of the receptor binding domain of NRG-2. In certain embodiments, EGF-like domain comprises the amino acid sequence of the receptor binding domain of NRG-3. In certain embodiments, EGF-like domain comprises the amino acid sequence of the receptor binding domain of NRG-4. In certain embodiments, EGF-like domain comprises the amino acid sequence of Ala Glu Lys Glu Lys Thr Phe Cys Val Asn Gly Gly Glu Cys Phe Met Val Lys Asp Leu Ser Asn Pro (SEQ ID NO:3), as described in U.S. Pat. No. 5,834,229.

As used herein, “protein” is synonymous with “polypeptide” or “peptide” unless the context clearly dictates otherwise.

As used herein, the terms “subject” and “patient” are used interchangeably. As used herein, a subject is preferably a mammal, such as a non-primate (e.g., cows, pigs, horses, cats, dogs, rats, etc.) or a primate (e.g., monkey and human), most preferably a human.

As used herein, “treat”, “treatment” and “treating” refer to any manner in which the symptoms of a condition, disorder or disease are ameliorated or otherwise beneficially altered. The effect may be prophylactic in terms of completely or partially preventing a disease or symptom thereof and/or may be therapeutic in terms of a partial or complete cure for a disease and/or adverse effect attributable to the disease. Treatment also encompasses any pharmaceutical use of the compositions herein.

As used herein, “activity unit” or “1 U” means the quantity of standard product that can induce 50% maximal reaction. In other words, to determine the activity unit for a given active agent, the EC50 must be measured. For example, if the EC50 for a batch of product was 0.067 μg/ml then that would be one unit. Further, if 1 μg of that product is being used then 14.93 U (1/0.067) is being used. The EC50 can be determined by any method known in the art, including the method employed by the inventors in the Examples below. This determination of the activity unit is important for quality control of genetically engineered products and clinically used drugs, permits product from different pharmaceuticals and/or different batch numbers to be quantified with uniform criteria.

In certain embodiments, unit of neuregulin is determined by measuring the activity of neuregulin through kinase receptor activation enzyme-linked immunosorbant assay (KIRA-ELISA) as described in detail in WO03/099300, and Sadick et al., 1996, Analytical Biochemistry, 235:207-14, the contents of which are incorporated by reference in their entireties. Briefly, the assay measures neuregulin induced ErbB2 activation and phosphorylation on the adherent breast carcinoma cell line, MCF-7. Membrane proteins are solubilized via Triton X-100 lysis and the receptor is captured in ELISA wells coated with ErbB2-specific antibodies (e.g., H4) with no cross-reaction to ErbB3 or ErbB4. The degree of receptor phosphorylation is then quantified by antiphosphotyrosine ELISA.

“Cardiomyogenesis,” as used herein, refers to the differentiation of progenitor or precursor cells into cardiac muscle cells (i.e., cardiomyocytes) and the growth of cardiac muscle tissue. Progenitor or precursor cells can be pluripotent stem cells such as, e.g., embryonic stem cells. Progenitor or precursor cells can be cells pre-committed to a myocardiac lineage (e.g., pre-cardiomyocyte cells) or cells that are not pre-committed (e.g., multipotent adult stem cells).

A “stem cell,” as used herein, refers to any self-renewing pluripotent cell or multipotent cell or progenitor cell or precursor cell that is capable of differentiating into multiple cell types. Stem cells suitable for use in the methods of the present invention include those that are capable of differentiating into cells of myocardiac lineage, e.g., cardiomyocytes. Suitable stem cells for use in the methods of the present invention include, for example, embryonic stem cells (“ESCs”) and embryonic carcinoma (“EC”) cells. Pluripotent embryonic stem cells are capable of differentiating into all types of tissue, including neuronal cells, muscle cells, blood cells, etc. See, e.g., Spradling et al., 2001, Nature 414:98-104.

“Differentiate” or “differentiation,” as used herein, refers to the process by which precursor or progenitor cells (i.e., stem cells) differentiate into specific cell types, e.g., cardiomyocytes. A differentiated cell can be identified by a number of features that are unique or distinctive with respect to that particular cell type. For example, differentiated cells may be identified by their patterns of gene expression and protein expression. Typically, cells of a myocardiac lineage express genes such as, for example, the sarcomeric myosin heavy chain, myosin light chain 2V, eHAND and ANF. See, e.g., Small et al., Cell, 110:725-735 (2002); Shin et al., Cell, 110:725-35 (2002). Also typically expressed by cells of a myocardiac lineage are cardiac muscle cell specific transcription factors such as MEF2, Nkx2.5 or the homeodomain transcription factor HOP. See, e.g., Edmondson et al., Development, 1251-1263 (1994); Lin et al., Science, 276:1404-1407 (1997). Additional transcription factors that are involved in cardiomyocyte differentiation include, e.g., GATA4 (see, e.g., Grepin et al., Development, 124:2387-95 (1997)). One skilled in the art will recognize that other cardiac muscle specific genes may be utilized to monitor and determine differentiation.

A “cardiomyocyte marker gene” is a gene which is expressed uniquely by developing cardiomyocytes or only rarely by other cell types, such that the marker gene is useful for the determination of whether a cell is a cardiomyocyte. An example of a cardiomyocyte marker gene is ANF, a polypeptide hormone that is synthesized primarily in cardiac myocytes and is a down-stream target of several cardiomyogenesis transcriptional factors.

A “solid support,” as used herein in connection with inducing cardiomyogenesis, refers to a three-dimensional matrix or a planar surface on which the stem cells can be cultured. The solid support can be derived from naturally occurring substances (i.e., protein based) or synthetic substances. For example, matrices based on naturally occurring substances may be composed of autologous bone fragments or commercially available bone substitutes as described in e.g., Clokie et al., J. Craniofac. Surg. 13(1): 111-21 (2002) and Isaksson, Swed. Dent. J. Suppl., 84:1-46 (1992). Suitable synthetic matrices are described in, e.g., U.S. Pat. Nos. 5,041,138, 5,512,474, and 6,425,222. For example, biodegradable artificial polymers, such as polyglycolic acid, polyorthoester, or polyanhydride can be used for the solid support. Calcium carbonate, aragonite, and porous ceramics (e.g., dense hydroxyapatite ceramic) are also suitable for use in the solid support. Polymers such as polypropylene, polyethylene glycol, and polystyrene can also be used in the solid support. Cells cultured and differentiated on a solid support that is a three-dimensional matrix typically grow on all of the surfaces of the matrix, e.g., internal and external. Cells cultured and differentiated on a solid support that is planar typically grow in a monolayer.

“Culturing,” as used herein, refers to maintaining cells under conditions in which they can proliferate and/or differentiate, and avoid senescence. For example, in the present invention, cultured embryonic stem cells can proliferate and differentiate into cells of a myocardiac cell lineage. Cells can be cultured in growth media containing appropriate growth factors, i.e., a growth factor cocktail containing proteins which facilitate or enhance the development of cardiomyocytes.

### 5.2 Use of Neuregulin to Induce Cardiomyogenesis

The present invention provides methods of inducing cardiomyogenesis in mammalian cells. In certain embodiments, the methods comprise the step of contacting a mammalian cell with neuregulin, whereupon the mammalian cell differentiates into a cell of a myocardiac lineage. The mammalian cell can be contacted with neuregulin or a composition thereof either in vivo or in vitro. For example, neuregulin could be administered directly to injured or malfunctioning cardiac muscle intravenously or by direct administration during surgery.

5.2.1 In Vivo Induction of Cardiomyogenesis

Neuregulin can be used to induce cardiomyogenesis in vivo. Neuregulin can be administered to a subject, e.g., a mammal such as a human, in an amount effective to induce differentiation of mammalian cells into cells of a myocardiac lineage. In view of its ability to induce cardiomyogenesis, neuregulin is believed to be useful for the repair of damaged myocardium in acute heart diseases and for treating disorders such as cardiomyopathy. In some embodiments, neuregulin can be used to generate cardiomyocytes for the purpose of studying the development of cardiac muscle tissue. In another embodiments, neuregulin can be used during the treatment of a subject in need of repair or augmentation of damaged or weakened cardiac muscle tissue. In another embodiments, neuregulin can be used to treat a subject who desires augmentation or enhancement of cardiac muscle tissue that is not damaged or weakened. Such subjects can include, for example, those at risk for cardiac diseases or disorders.

One of skill in the art will appreciate that neuregulin can be can be used alone or in combination with other compounds and therapeutic regimens to induce cardiomyogenesis. For example, neuregulin can be may be administered in conjunction with purified or synthesized growth factors and other agents, or combinations thereof, which enhance the development of cardiac muscle tissue.

An effective amount of neuregulin can be determined by, for example, the existence, nature, and extent of any adverse side-effects that accompany the administration of the composition; the EC50 of neuregulin; and the side-effects of neuregulin at various concentrations. Typically, the amount of neuregulin administered will range from about 0.001 to about 20 mg per kg, more typically about 0.05 to about 15 mg per kg, even more typically about 0.01 to about 10 mg per kg body weight.

Neuregulin can be administered, for example, by intravenous infusion, orally, intraperitoneally, or subcutaneously. Intravenous administration is the preferred method of administration. The formulations of neuregulin can be presented in unit-dose or multi-dose sealed containers, such as ampoules and vials.

Neuregulin can be typically formulated with a pharmaceutically acceptable carrier before administration to an individual or subject. Pharmaceutically acceptable carriers are determined, in part, by the particular composition being administered as well as by the particular method used to administer the composition. Accordingly, there are a wide variety of suitable formulations of pharmaceutical compositions of the present invention (see, e.g., Remington's Pharmaceutical Sciences, 17th ed., 1989).

Neuregulin may be may be in formulations suitable for other routes of administration, such as, for example, intravenous infusion, orally, intraperitoneally, or subcutaneously. The formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can be prepared from sterile powders, granules, and tablets.

The dose administered to a subject, in the context of the present invention should be sufficient to effect a beneficial therapeutic response in the patient over time. For example, if neuregulin is administered to treat or prevent cardiomyopathy, the dose administered to the patient should be sufficient to prevent, retard, or reverse the diminished capacity of the cardiac muscle to rhythmically contract. The dose will be determined by the efficacy of the particular composition employed and the condition of the patient, as well as the body weight or surface area of the patient to be treated. The size of the dose also will be determined by the existence, nature, and extent of any adverse side-effects that accompany the administration of a particular composition in a particular patient.

### 5.2.2 In Vitro Induction of Cardiomyogenesis

Neuregulin can conveniently be used to induce cardiomyogenesis in vitro. In certain embodiments, mammalian cells are contacted with neuregulin, whereupon the mammalian cells differentiates into cells of a myocardiac lineage.

The cells to be differentiated into cells of a myocardiac lineage can be derived from any suitable mammal. For example, the cells can be obtained from rodents such as, for example, mice, rats, guinea pigs, and rabbits; non-rodent mammals such as, for example, dogs, cats, pigs, sheep, horses, cows, and goats; primates such as, for example, chimpanzees and humans. The cells to be differentiated may be primary cells or may be cells maintained in culture. If the cells are maintained in culture, they are typically contacted with neuregulin between the 12th and 15th passage in culture. Techniques and methods for establishing a primary culture of cells for use in the methods of the invention are known to those of skill in the art (see, e.g., Humason, ANIMAL TISSUE TECHNIQUES, 4^{th} ed., W. H. Freeman and Company (1979), and Ricciardelli et al., In vitro Cell Dev. Biol., 25:1016 (1989)).

The stem cells contacted by NRG can be human stem cells, for example, human mesenchymal stem cells (MSC). MSC may be obtained by isolating pluripotent mesenchymal stem cells from other cells in the bone marrow or other MSC source. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, blood, adipose tissue, and muscle satellite cells. Typically, cells from a tissue specimen containing mesenchymal stem cells are cultured in growth medium containing growth factors that (1) stimulate mesenchymal stem cell growth without differentiation, and (2) allow for the selective adherence of only the mesenchymal stem cells to a substrate surface. After culturing the cells for a suitable amount of time, non-adherent matter is removed from the substrate surface, thus providing an expanded population of mesenchymal stem cells. Thus, homogeneous MSC populations are obtained by positive selection of adherent marrow or periosteal cells which are free of markers associated with either hematopoietic cell or differentiated mesenchymal cells.

Preferably, the mammalian cells contacted by neuregulin are stem cells, particularly embryonic stem cells (ESCs). Methods for isolation of human and animal ESCs are well known in the art. See, e.g., Brook F A, Proc. Natl. Acad. Sci. USA, 94:5709-12 (1997); Grounds et al., J. Histochem. and Cytochem., 50:589-610 (2002); Reubinoff, Nat. Biotech., 18:399-404 (2000). Mammalian embryonic stem cells include, for example, murine R1 cells and human embryonic stem cells.

The mammalian cells (e.g., ESCs) may be contacted with neuregulin alone, in combination with, either together in a single mixture or sequentially, or in the presence of other growth factors. Those of skill in the art will appreciate that the amount of neuregulin and growth factors can be adjusted to facilitate induction of differentiation in particular cell types.

Cells can be cultured according to routine techniques in the field of cell culture. Suitable cell culture methods and conditions can be determined by those of skill in the art using known methodology (see, e.g., Freshney et al., CULTURE OF ANIMAL CELLS (3^{rd} ed. 1994)). In general, the cell culture environment includes consideration of such factors as the substrate for cell growth, cell density and cell contract, the gas phase, the medium, and temperature.

Incubation of cells is generally performed under conditions known to be optimal for cell growth. Such conditions may include, for example, a temperature of approximately 37° C. and a humidified atmosphere containing approximately 5% CO₂. The duration of the incubation can vary widely, depending on the desired results. In general, incubation is preferably continued until the cells express suitable Proliferation is conveniently determined using ³H thymidine incorporation or BrdU labeling.

Plastic dishes, flasks, or roller bottles may be used to culture cells according to the methods of the present invention. Suitable culture vessels include, for example, multi-well plates, Petri dishes, tissue culture tubes, flasks, roller bottles, and the like.

Cells can be grown at optimal densities that are determined empirically based on the cell type. Cells are typically passaged 12-15 times and discarded after 15 passages.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Important constituents of the gas phase are oxygen and carbon dioxide. Typically, atmospheric oxygen tensions are used for cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration typically is 5%.

Defined cell media are available as packaged, premixed powders or presterilized solutions. Examples of commonly used media include MEM-α, DME, RPMI 1640, DMEM, Iscove's complete media, or McCoy's Medium (see, e.g., GibcoBRL/Life Technologies Catalogue and Reference Guide; Sigma Catalogue). Typically, MEM-α or DMEM are used in the methods of the invention. Defined cell culture media are often supplemented with 5-20% serum, typically heat inactivated serum, e.g., human, horse, calf, and fetal bovine serum. Typically, 10% fetal bovine serum is used in the methods of the invention. The culture medium is usually buffered to maintain the cells at a pH preferably from about 7.2 to about 7.4. Other supplements to the media typically include, e.g., antibiotics, amino acids, and sugars, and growth factors.

5.2.3 Detection of Cardiomyogenesis

After administration neuregulin in vivo or in vitro, the induction of cardiomyogenesis can be detected by a number of different methods including, but not limited to: detecting expression of cardiomyocyte-specific proteins, detecting expression of cardiac muscle cell-specific transcription factors, detecting expression of proteins essential for cardiac muscle function, and detecting the beating of cardiac muscle cells.

5.2.4 Administration of Differentiation Cardiomyocytes

Differentiated cardiomyocytes can be administered to a subject by any means known to those of skill in the art. In one embodiment of the invention, differentiated cardiomyocytes on an intact solid support (e.g., a three-dimensional matrix or a planar surface) can be administered to the subject, e.g., via surgical implantation. Alternatively, the differentiated cardiomyocytes can be detached from the matrix, i.e., by treatment with a protease, before administration to the subject, e.g., intravenous, subcutaneous, or intraperitoneal.

In some embodiments, embryonic stem cells are extracted and subsequently contacted with a matrix for proliferation and differentiation into cells of a myocardiac lineage. Cells can be extracted from the subject to be treated, i.e., autologous (thereby avoiding immune-based rejection of the implant), or can be from a second subject, i.e., heterologous. In either case, administration of cells can be combined with an appropriate immunosuppressive treatment.

Cardiomyocytes differentiated according to the methods of the present invention may be administered to a subject by any means known in the art. Suitable means of administration include, for example, intravenous, subcutaneous, intraperitoneal, and surgical implantation. The cardiomyocytes may be directly injected into cardiac muscle or applied topically, for example, during surgery on the heart.

The cells may be in formulations suitable for administration, such as, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can be prepared from sterile powders, granules, and tablets.

For surgical implantation, differentiated cells are typically left on an intact solid support, e.g., a three-dimensional matrix or planar surface. The matrix or planar surface can be surgically implanted into the appropriate site in a subject. For example, a patient needing a replacement of a portion of cardiac muscle tissue can have differentiated cells on an intact solid support surgically implanted.

In determining the effective amount of the cells to be administered in the treatment or prophylaxis of conditions owing to diminished or malfunctioning cardiac muscle cells, the physician evaluates cell toxicity, transplantation reactions, progression of the disease, and the production of anti-cell antibodies. For administration, cardiomyocytes differentiated according to the methods of the present invention can be administered in an amount effective to provide cardiac muscle cells to the subject, taking into account the side-effects of the cardiomyocytes at various concentrations, as applied to the mass and overall health of the patient. Administration can be accomplished via single or divided doses.

5.3 Neuregulin

Any neuregulin (e.g., NRG-1, NRG-2, NRG-3 and NRG-4 and isoforms thereof) protein, peptide or fragment can be used in the present invention.

Neuregulin or NRG refers to proteins or peptides that can bind and activate ErbB2, ErbB3, ErbB4 or combinations thereof, including but not limited to all neuregulin isoforms, neuregulin EGF domain alone, polypeptides comprising neuregulin EGF-like domain, neuregulin mutants or derivatives, and any kind of neuregulin-like gene products that also activate the above receptors as described in detail below. In preferred embodiments, neuregulin used in the present invention binds to and activate ErbB2/ErbB4 or ErbB2/ErbB3 heterodimers. Neuregulin used in the present invention can activate the above ErbB receptors and modulate their biological reactions, e.g., stimulate breast cancer cell differentiation and milk protein secretion; induce the differentiation of neural crest cell into Schwann cell; stimulate acetylcholine receptor synthesis in skeletal muscle cell; and/or improve cardiocyte differentiation, survival and DNA synthesis. Assays for measuring the receptor binding activity are known in the art. For example, cells transfected with ErbB-2 and ErbB-4 receptor can be used. After receptor expressing cells are incubated with excess amount of radiolabeled neuregulin, the cells are pelleted and the solution containing unbound radiolabeled neuregulin is removed before unlabeled neuregulin solution is added to compete with radiolabeled neuregulin. EC50 is measured by methods known in the art. EC50 is the concentration of ligands which can compete 50% of bound radiolabeled ligands off the receptor complex. The higher the EC50 value is, the lower the receptor binding affinity is.

Neuregulin used in the present invention includes any neuregulin and isoforms thereof known in the art, including but not limited to all isoforms of neuregulin-1 (“NRG-1”), neuregulin-1 (“NRG-2”), neuregulin-1 (“NRG-3”) and neuregulin-4 (“NRG-43”). NRG-1 is described, for example, in U.S. Pat. Nos. 5,530,109, 5,716,930, and 7,037,888; Lemke, Mol. Cell. Neurosci. 1996, 7:247-262; Peles and Yarden, 1993, BioEssays 15:815-824, 1993; Peles et al., 1992, Cell 69, 205-216; Wen et al., 1992, Cell 69, 559-572, 1992, Holmes et al., 1992, Science 256:1205-1210, Falls et al., 1993, Cell 72:801-815, Marchionni et al. 1993, Nature 362:312-8, the contents of which are incorporated by reference in their entireties. NRG-2 is described, for example, in Chang et al., 1997, Nature 387:509-512; Carraway et al., 1997, Nature 387:512-516; Higashiyama et al., 1997, J. Biochem. 122:675-680, Busfield et al., 1997, Mol. Cell. Biol. 17:4007-4014 and International Pat. Pub. No. WO 97/09425), the contents of which are incorporated by reference in their entireties. NRG-3 is described, for example, in Hijazi et al., 1998, Int. J. Oncol. 13:1061-1067, the contents of which are incorporated by reference in their entireties. NRG-4 is described, for example, in Harari et al., 1999 Oncogene. 18:2681-89, the contents of which are incorporated by reference in their entireties.

Neuregulin used in the present invention includes neuregulin mutants or derivatives that comprise one or more amino acid substitutions, deletions, and/or additions that are not present in the naturally occurring neuregulin. Preferably, the number of amino acids substituted, deleted, or added is 1, 2, 3, 4, 5, 6, 7, 8, 9, or 10 amino acids. In one embodiment, such a derivative contains one or more amino acid deletions, substitutions, or additions at the amino and/or carboxy terminal end of the peptide. In another embodiment, such a derivative contains one or more amino acid deletions, substitutions, or additions at any residue within the length of the peptide. Exemplary neuregulin variants are described in WO2007/06254, the contents of which are incorporated by reference in their entirety.

In certain embodiments, the amino acid substitutions may be conservative or non-conservative amino acid substitutions. Conservative amino acid substitutions are made on the basis of similarity in polarity, charge, solubility, hydrophobicity, hydrophilicity, and/or the amphipathic nature of the amino acid residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for another class.

In certain embodiments, neuregulin used in the present invention is a neuregulin derivative with conservative amino acid substitutions that do not substantially alter their biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In certain embodiments, neuregulin used in the present invention includes neuregulin mutants or derivatives having an amino acid substitution with a non-classical amino acid or chemical amino acid analog. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Neuregulin used in the present invention includes neuregulin homologue, that is, a polypeptide that exhibits an amino acid sequence homology and/or structural resemblance to neuregulin, or to one of the interacting domains of neuregulin such that it is capable of bind and activate ErbB2/ErbB4 or ErbB2/ErbB3 heterodimers protein kinases. Typically, a protein homologue of a native protein may have an amino acid sequence that is at least 50%, preferably at least 75%, more preferably at least 80%, 85%, 86%, 87%, 88% or 89%, even more preferably at least 90%, 91%, 92%, 93% or 94%, and most preferably 95%, 96%, 97%, 98% or 99% identical to the native protein.

Percent homology in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology. In certain embodiments, neuregulin homologue is characterized by its percent sequence identity or percent sequence similarity with the naturally occurring neuregulin sequence. Sequence homology, including percentages of sequence identity and similarity, are determined using sequence alignment techniques well-known in the art, preferably computer algorithms designed for this purpose, using the default parameters of said computer algorithms or the software packages containing them.

Nonlimiting examples of computer algorithms and software packages incorporating such algorithms include the following. The BLAST family of programs exemplify a preferred, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another preferred example is the algorithm of Myers and Miller (1988 CABIOS 4:11-17) which is incorporated into the ALIGN program (version 2.0) and is available as part of the GCG sequence alignment software package. Also preferred is the FASTA program (Pearson W. R. and Lipman D. J., Proc. Nat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Needleman and Wunsch (J. Mol. Biol. 48:443-354, 1970), and is preferable where the two sequences are approximately the same length and an alignment is expected over the entire length.

Examples of homologues may be the ortholog proteins of other species including animals, plants, yeast, bacteria, and the like. Homologues may also be selected by, e.g., mutagenesis in a native protein. For example, homologues may be identified by site-specific mutagenesis in combination with assays for detecting protein-protein interactions. Additional methods, e.g., protein affinity chromatography, affinity blotting, in vitro binding assays, and the like, will be apparent to skilled artisans apprised of the present invention.

For the purpose of comparing two different nucleic acid or polypeptide sequences, one sequence (test sequence) may be described to be a specific “percent identical to” another sequence (reference sequence) in the present disclosure. In this respect, when the length of the test sequence is less than 90% of the length of the reference sequence, the percentage identity is determined by the algorithm of Myers and Miller, Bull. Math. Biol., 51:5-37 (1989) and Myers and Miller, Comput. Appl. Biosci., 4(1):11-17 (1988). Specifically, the identity is determined by the ALIGN program. The default parameters can be used.

Where the length of the test sequence is at least 90% of the length of the reference sequence, the percentage identity is determined by the algorithm of Karlin and Altschul, Proc. Natl. Acad. Sci. USA, 90:5873-77 (1993), which is incorporated into various BLAST programs. Specifically, the percentage identity is determined by the “BLAST 2 Sequences” tool. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter).

Neuregulin used in the present invention also include neuregulin EGF domain alone, polypeptides comprising neuregulin EGF domain or neuregulin-like gene products that mimic the activities of neuregulin and binds and activates ErbB2, ErbB3, ErbB4 or combinations thereof. As used herein, “epidermal growth factor-like domain” or “EGF-like domain” refers to a polypeptide motif encoded by the neuregulin gene that binds to and activates ErbB2, ErbB3, ErbB4, or combinations thereof, and bears a structural similarity to the EGF receptor-binding domain as disclosed in WO 00/64400, Holmes et al., Science, 256:1205-1210 (1992); U.S. Pat. Nos. 5,530,109 and 5,716,930; Hijazi et al., Int. J. Oncol., 13:1061-1067 (1998); Chang et al., Nature, 387:509-512 (1997); Carraway et al., Nature, 387:512-516 (1997); Higashiyama et al., J. Biochem., 122:675-680 (1997); and WO 97/09425, the contents of which are all incorporated herein by reference.

In certain embodiments, neuregulin used in the present invention comprises the EGF-like domain encoded by NRG-1. In some embodiments, EGF-like domain comprises the amino acid sequence of the receptor binding domain of NRG-1. In some embodiments, EGF-like domain comprises the amino acid sequence corresponding to amino acid residues 177-226, 177-237, or 177-240 of NRG-1.

In preferred embodiments, neuregulin used in the present invention comprises the amino acid sequence of: Ser His Leu Val Lys Cys Ala Glu Lys Glu Lys Thr Phe Cys Val Asn Gly Gly Glu Cys Phe Met Val Lys Asp Leu Ser Asn Pro Ser Arg Tyr Leu Cys Lys Cys Pro Asn Glu Phe Thr Gly Asp Arg Cys Gln Asn Tyr Val Met Ala Ser Phe Tyr Lys Ala Glu Glu Leu Tyr Gln (SEQ ID NO:1), which corresponds to amino acids 177-237 of human NRG-1. The human nucleic acid sequence encoding the fragment is: agccatcttg taaaatgtgc ggagaaggag aaaactttct gtgtgaatgg aggggagtgc ttcatggtga aagacctttc aaacccctcg agatacttgt gcaagtgccc aaatgagttt actggtgatc gctgccaaaa ctacgtaatg gcgagcttct acaaggcgga ggagctgtac cag (SEQ ID NO:2).

In certain embodiments, neuregulin used in the present invention comprises the EGF-like domain encoded by NRG-2. In certain embodiments, neuregulin used in the present invention comprises the EGF-like domain encoded by NRG-3. In certain embodiments, neuregulin used in the present invention comprises the EGF-like domain encoded by NRG-4. In certain embodiments, neuregulin used in the present invention comprises the amino acid sequence of Ala Glu Lys Glu Lys Thr Phe Cys Val Asn Gly Gly Glu Cys Phe Met Val Lys Asp Leu Ser Asn Pro (SEQ ID NO:3) as described in U.S. Pat. No. 5,834,229.

Neuregulin for administration in the methods of the present invention can be in any neuregulin formulation or composition apparent to those of skill in the art. Exemplary pharmaceutical formulations and compositions are described in for example, in U.S. Pat. No. 7,226,907, U.S. Pat. No. 5,367,060, WO 94/026298, WO 03/099300, the contents of each of which are incorporated by reference in its entirety.

Neuregulin can be prepared, formulated and administered to a subject by any methods apparent to those of skill in the art as described below.

5.3.1 Preparation of Neuregulin

Neuregulin can be prepared according to any technique apparent to those of skill. Exemplary techniques for the preparation of neuregulin are described, for example, in U.S. Pat. No. 7,226,907, U.S. Pat. No. 5,367,060, WO 94/026298, WO 03/099300, the contents of which are hereby incorporated by reference in its entirety. In certain embodiments, neuregulin can be prepared synthetically, for example by solution phase or solid phase peptide synthesis. See Merrifield, 1963, J. Am. Chem. Soc. 85:2149; Fields et al., 1990, Int J Pept Protein Res. 35:161-214; Fields et al., 1991, Pept Res. 4:95-101; the contents of which are hereby incorporated by reference in their entirety.

In further embodiments, neuregulin can be obtained from natural sources, recombinant sources or commercial sources. In some embodiments, neuregulin can be obtained by recombinantly expressing procolipase, cleaving procolipase to produce neuregulin and then purifying neuregulin.

Neuregulin used in the present invention can be purified by any art-known technique such as high performance liquid chromatography, ion exchange chromatography, gel electrophoresis, affinity chromatography and the like. The actual conditions used to purify a particular neuregulin will be apparent to those having skill in the art.

### 6. EXAMPLES

### 6.1 Example 1

### Effect of Neuregulin on Regeneration of Heart

Experimental methods:

1. Rat left ventricle coronary artery ligation and echocardiography

Wistar male rats (Shanghai Animal Center of Chinese Academy of Science), which weighed 200±20 g, were anesthetized by intraperitoneally injecting 100 mg/kg (drug/body weight) of ketamine. The neck and chest were depilated and sanitized. An incision was made in the middle front neck to expose the tracheae. An 18 G catheter overneedle was inserted into the tracheae between the 3rd and 5th cartilage of tracheae. After the needle was drawn out, a plastic cannula was pushed into the trachea 1-2 cm and fixed to connect the Rodent Ventilator (SAR-830/P ventilator—Inspiratory flow rate, 1 ml/100 g/breath; Respiratory rate, 60 breaths/min). Another incision was made on the left front chest. The skin was blunt dissected to expose the fourth and fifth rib, then the fourth rib was cut by elbowed mosquito forceps. The ventilator (as described above) was linked to the cannula and turned on, and the heart was exposed to check the status of lung and heart. The pericardium was rived off to identify the left atria and the pulmonary arterious cone after the heart was exteriorized through the incision. The left ventricle anterior descending coronary artery between them was ligated tight with 6/0 medical suture before the heart was replaced into the thorax. The thoracic wall was stitched. The ventilator was blocked to full fill the lung. The chest muscle and skin was stitched after the air in the thoracic cavity was gently squeezed out. The ventilators were removed from the rats until constant spontaneous respiration resumed.

The cardiac function of the rats was then examined by echocardiography (Philips Sonos 7500 S4 probe) on the 14th day after ligation. The rats with ejection fraction (hereinafter “EF”) values from 30 to 50 percent were separated and grouped (15 rats per group).

Results

The rats were weighed on the 15th day after left ventricle coronary ligation to determine the amount of vehicle or NRG needed. Rats in the vehicle group received 0.4 ml/100 g (volume/body weight) of vehicle (10 mM Na₂HPO4-NaH₂PO4, 150 mM NaCl, 0.2% HSA (human serum albumin), 5% mannitol, pH 6.0) by IV injection, while rats in the neuregulin group received 0.4 ml/100 g (volume/body weight) of neuregulin (1.625 μg/ml, which is about 24.26 U/ml) by IV injection. The vehicle or neuregulin was injected once a day for five days, stopped for two days, and then injected for another five days. Then the cardiac function of all rats was checked again by echocardiography (Philips Sonos 7500 S4 probe).

After another 2 days, the rats were sacrificed by IV injection with 3-5 ml 10% KCl, then the hearts were taken and washed before fixed in 10% formalin for 15-20 minutes. The hearts were then weighed, and were further sectioned (Leica, BM2135), stained by hematoxylin and eosin before pictures were taken (1.5×).

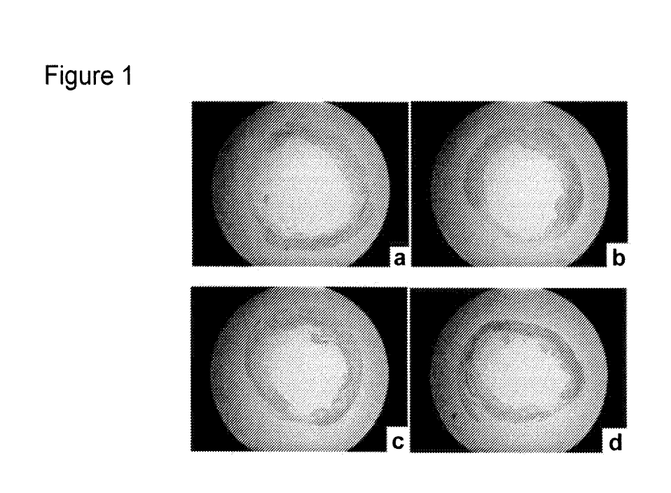

As shown in FIG. 1 of the pictures of stained heart section, the infarct area in FIG. 1c or 1d (infarcted heart treated with neuregulin) is much smaller than that in FIG. 1a or 1b (infarcted heart treated with vehicle). The average percentage of infarct area (100%×infarct area/total area) for injured heart treated with neuregulin (12.30±4.6) is also smaller than that for injured heart treated with vehicle (17.87±4.7). This demonstrates that the neuregulin induces angiogenesis and tissue regeneration in the injured heart.

The scope of the invention is not limited by the description of the examples. Modifications and alterations of the present invention will be apparent to those skilled in the art without departing from the scope and spirit of the present invention. Therefore, it will be appreciated that the scope of this invention is to be defined by the appended claims, rather than by the specific examples which have been presented by way of example.

What is claimed is:

1. A method of inducing myocardiogenesis, the method comprising: contacting a mammalian cell with neuregulin, whereby the mammalian cell differentiates into a cell of myocardiac lineage.

2. The method of claim 1, wherein the neuregulin is neuregulin 1, neuregulin 2, neuregulin 3 or neuregulin 4.

3. The method of claim 1, wherein the neuregulin comprises an EGF like domain.

4. The method of claim 1, wherein neuregulin comprises the amino acid sequence of SEQ ID NO:1.

5. The method of claim 1 wherein said neuregulin is in a pharmaceutically acceptable carrier.

6. The method of claim 1, wherein the mammalian cell is in a mammal.

7. The method of claim 6, wherein the step of contacting is by intravenous administration of neuregulin to the mammal.

8. The method of claim 1, further comprising detecting differentiation of the mammalian cell into a cell of a myocardiac lineage.

9. The method of claim 8, whereby differentiation of the mammalian cell into a cell of a myocardiac lineage is detected by detecting expression of a cardiomyocyte marker gene.

10. The method of claim 8, whereby differentiation of the mammalian cell into a cell of a myocardiac lineage is detected by detecting expression of a cardiac muscle cell-specific transcription factor.

11. The method of claim 8, whereby differentiation of the mammalian cell into a cell of a myocardiac lineage is detected by detecting expression of a cardiac specific gene.

12. The method of claim 1, wherein the mammalian cell is an embryonic stem cell.

13. The method of claim 1, wherein the mammalian cell is a primate embryonic stem cell.

14. The method of claim 1, wherein the mammalian cell is a human embryonic stem cell.

15. The method of claim 1, wherein the mammalian cell is further contacted with a cardiomyogenesis enhancing protein.

16. The method of claim 15, wherein the cardiomyogenesis enhancing protein is a growth factor involved in cardiomyogenesis.

17. The method of claim 1, wherein the mammalian cell is attached to a solid support.

18. The method of claim 17, wherein the solid support is a three dimensional matrix.

19. A method of treating a cardiac muscle disorder, the method comprising: contacting a mammalian cell with neuregulin, whereby the mammalian cell differentiates into a cell of a myocardiac lineage.

20. The method of claim 19, wherein the neuregulin is neuregulin 1, neuregulin 2, neuregulin 3 or neuregulin 4.

21. The method of claim 19, wherein the neuregulin comprises an EGF like domain.

22. The method of claim 19, wherein neuregulin comprises the amino acid sequence of SEQ ID NO:1.

23. The method of claim 19, wherein the cardiac muscle disorder is associated with damaged myocardium.

24. The method of claim 23, wherein the cardiac muscle disorder is heart failure.

25. The method of claim 19, further comprising administering the cell of a myocardiac lineage to an individual with the disorder, thereby treating the disorder.

26. The method of claim 25, wherein the administration is by surgical implantation.


SEQUENCE LISTING (3 sequences)

SEQ ID NO: 1 - protein, 61 aa, Homo sapiens
SHLVKCAEKEKTFCVNGGECFMVKDLSNPSRYLCKCPNEFTGDRCQNYVMASFYKAEELYQ

SEQ ID NO: 2 - DNA, 183 bp, Homo sapiens
agccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggcgagcttctacaaggcggaggagctgtaccag

SEQ ID NO: 3 - protein, 23 aa, Homo sapiens
AEKEKTFCVNGGECFMVKDLSNP